(19) United States
(12) Patent Application Publication
(10) Pub. No.: US 2022/0273215 A1
(43) Pub. Date: Sep. 1, 2022

(54) SYSTEM AND METHOD FOR ASSESSING ABDOMINAL MUSCLE STRENGTH

(71) Applicant: Daniel SKINNER, Victoria (CA)

(72) Inventor: Daniel SKINNER, Victoria (CA)

(21) Appl. No.: 17/684,073

(22) Filed: Mar. 1, 2022

(30) Foreign Application Priority Data
Mar. 1, 2021 (CA) 3110793

(51) Int. Cl. A61B 5/22; A61B 5/00

(52) U.S. Cl. CPC A61B 5/224; A61B 5/6831; A61B 5/6823; A61B 5/742; A61B 5/0022; A61B 2562/0247; A61B 2562/043

(57) ABSTRACT

A pressure monitor is provided for reporting pressure exerted by each of a plurality of core muscles of a user, the pressure monitor comprising: an adjustable belt, the adjustable belt including an inner surface and an outer surface; a plurality of pressure sensors movably mounted on the inner surface of the adjustable belt; a microcontroller which is mounted on the belt and is in electronic communication with each of the plurality of pressure sensors; and a user interface, the user interface in communication with the microcontroller.

### CROSS REFERENCE TO RELATED APPLICATIONS

The present invention is related to and claims the benefit of Canadian Patent Application Serial No. 3,110,793, filed on Mar. 1, 2021, and entitled SYSTEM AND METHOD FOR ASSESSING ABDOMINAL MUSCLE STRENGTH and which is hereby incorporated in its entirety including all tables, figures, and claims.

### FIELD

The present technology is directed to providing information on the core strength of a patient or user based on biofeedback from individual muscles of the abdomen. More specifically it is a belt with a plurality of sensors in communication with a user interface for measuring intra-abdominal pressure.

### BACKGROUND

Maintenance of core strength is important for maintaining correct posture and to avoid back injuries. The core is made up of the muscles in the abdomen, lower back, and pelvis that lie roughly between the rib cage and the hips. The strength and coordination of these muscles is important not only for sports and fitness routines but also for daily life. Thus, the core as a whole is made of a number of muscles which work at varying levels of engagement. A fit core has all the core muscles contributing. Thus, measurement of only one muscle or a gross measurement of the entire core does not provide useful information for a user, a patient or a practitioner. Examples of gross measurements of abdominal strength include United States Patent Application Publication No. 20190269544 which discloses methods and systems of reducing, treating, and/or preventing back pain in an individual, such as a pregnant or post-partum individual, that include changing the center of gravity of the individual by the methods set forth herein. In particular, the methods herein include shifting the center of gravity in the individual to a desired center of gravity. The methods include applying modifiable lifting forces to a lower abdomen of the individual using a pully system, under the belly of the individual toward the desired center of gravity; and applying one or more vector forces from an anterior side of the individual toward the desired center of gravity. Also provided are methods that include applying a lumbar support, as described herein to an individual. Also provided herein are adjustable, modular lumbar support devices that may be used to provide lumbar and/or belly support for individuals having belly weight to achieve the present methods. According to non-limiting example embodiments, support or brace devices provided herein may be worn by pregnant women at various stages throughout a woman's pregnancy, and/or post-partum and may be adjusted to naturally contour/conform to the woman's anatomy, which changes throughout pregnancy. Further provided are kits that include at least one lumbar support device provided herein, which kits may optionally include instructions for proper application and/or adjustment of the lumbar support device and/or accessories for use with the present methods, systems and/or devices. Pressure sensors are located under the brace to report on the pressure being exerted by the user's abdomen and by the brace. A remote computing device is in Bluetooth® communication with the sensors. The brace exerts the pressure and therefore the measurements are not indicative of the patient's abdominal strength.

U.S. Pat. No. 10,716,507 discloses a palpation assisting apparatus includes a synchronization controller configured to synchronize a timing of acquiring pressure data output from a pressure sensor attached to a position in contact with a lesion part with a timing of acquiring positional information of a marker attached to a position corresponding to the pressure sensor, and an entering part configured to store, in a storage part, the pressure data and the positional information of the marker that are acquired at the synchronized timing in association with time information indicating a time at which the pressure data and the positional information are acquired. The apparatus relies on the health care professional manually palpating the patient. Sensing abdominal strength with the health care professional and the apparatus is disclosed. This relies on the health care professional to exert the pressure and the pressure is limited to one location at a time.

U.S. Pat. No. 7,833,142 discloses that an abdominal mat has a raised section which may be positioned to support the lumbar region of the user's back. At least one sensor is associated with the raised section for producing a signal in response to force applied to the raised section by the user. A feedback device is coupled to the sensor for providing feedback to the user based on the force applied to the raised section. A user may use the device to test the strength of his or her abdominal muscles, and/or to monitor the force applied to the raised section of the mat when the user is exercising his or her abdominal muscles. The raised section may comprise an inflatable bladder connected to be automatically inflated and deflated by an electronic pump to accommodate different body types, therefore establishing a consistent starting point. This mat will only measure the force exerted on the raised section and does not truly measure abdominal strength. Further, it is not able to provide information on the strength of specific muscles of the abdomen.

WO2011007101 discloses an apparatus (1) for evaluating the muscular capacity of the abdominal muscles of a patient, including: a means (10) to be tightened around the abdominal muscles of the patient; a means (12, 13) for measuring the abdominal pressure of the patient connected to the tightening means (10); a means (14) for monitoring the means (12, 13) for measuring the abdominal pressure, designed to a) determine the mean abdominal pressure (PM) of the patient over a sequence of respiratory movements by the patient; b) determine an abdominal pressure, referred to as cough pressure (PT), over a sequence of coughs by the patient; c) evaluate muscular capacity on the basis of the orientation of the abdominal cough pressure (PT) and of the result of the comparison between the abdominal cough pressure (PT) and the mean abdominal pressure (PM). The invention also relates to a kit for abdominal muscle training and rehabilitation including such an apparatus. The apparatus includes a fluid retaining bag, which is preferably an air bag that is attached to a belt. There are pressure sensors in the air bag. It is not able to provide information on the strength of specific muscles of the abdomen.

United States Patent Application Publication No. 20050170938 discloses a belt with an inflatable bladder which, when inflated, is permitted to expand toward an interior of the belt and prevented by a barrier from expanding toward an exterior of the belt. A pressure gauge indicates the pressure within the bladder, and the gauge is fixedly displaced relative to the belt and the user such that the gauge may be viewed by a user when the belt is worn without significantly moving the cervical spine substantially out of a neutral posture. The strength of the transverse abdominal muscles is measured with the belt. It is not able to provide information on the strength of specific muscles of the abdomen.

WO2016204631 discloses a pressure sensor apparatus that includes: a plurality of pressure responsive chambers provided along a longitudinal dimension of the apparatus, and a pressure sensor device provided in each chamber which together provide a pressure profile in an anatomical cavity. This is specifically for internal use only.

U.S. Pat. No. 5,823,913 discloses a device for exercising the abdominal muscles and method comprising an informing unit having a sensor, an announciator with a signal-controlling circuit board and a signal reproducer, and elements to enable positioning and retaining the informing unit on the user's belly. The abdominal muscles are exercised by flexing them using the information from the sensor. These attract the user's attention to the exercising of abdominal muscles, by reproducing an exercise command or musical note, serving to indicate to the user to contract muscles, and stop the command, therefore resulting in more efficient development of the abdominal muscles. It is not able to provide information on the strength of specific muscles of the abdomen.

United States Patent Application Publication No. 20160199696 discloses a system and method for development of core muscles' support, comprising a means for identifying a user qualifying movement, a means for detecting a core muscle contraction in the identified qualifying movement, a means for discriminating between a core muscle contraction and no core muscle contraction in the identified qualifying movement; and a means to provide feedback to the user. It is not able to provide information on the strength of specific muscles of the abdomen.

What is needed is a system that monitors strength of individual muscles of the abdomen to provide a comprehensive assessment of core strength and to identify muscles that are weaker or are not engaging properly. It would be preferable if the system included a belt with a plurality of pressure sensors that can be positioned over the muscles of interest. It would be preferable if the belt included a microcontroller with a wireless communication link to a computing device or included a microcontroller and a display. It would be further preferable if the display was a user interface or a plurality of red-blue-green light emitting diode lights.

### SUMMARY

The present technology is a system that monitors strength of individual muscles of the abdomen to provide a comprehensive assessment of core strength and to identify muscles that are weaker or are not engaging properly. The system includes a belt with a plurality of pressure sensors that can be positioned over the muscles of interest. In one embodiment, the belt includes a microcontroller with a wireless communication link to a computing device. In another embodiment, the belt includes a microcontroller and a display. In one embodiment, the display is a user interface. In another embodiment, the display is a plurality of red-blue-green (RBG) light emitting diode lights.

In one embodiment a pressure monitor for reporting pressure exerted by each of a plurality of core muscles of a user is provided, the pressure monitor comprising: an adjustable belt, the adjustable belt including an inner surface and an outer surface; a plurality of pressure sensors movably mounted on the inner surface of the adjustable belt; a microcontroller which is mounted on the belt and is in electronic communication with each of the plurality of pressure sensors; and a user interface, the user interface in communication with the microcontroller.

In the pressure monitor, the user interface may be mounted on the belt.

In the pressure monitor, the microcontroller may be configured to receive pressure data from each of the plurality of pressure sensors, display the pressure data on the user interface and store the pressure data.

The pressure monitor may further comprise a computing device which is in wireless communication with the microcontroller and includes the user interface.

In the pressure monitor, the computing device may be configured to receive pressure data from each of the plurality of pressure sensors, display the pressure data on the user interface and store the pressure data.

The pressure monitor may further comprise a slide on the inner surface of the belt, the plurality of pressure sensors slidably mounted on the slide.

In the pressure monitor, the belt may further comprise an inner layer, an outer layer and a pocket therebetween.

The pressure monitor may further comprise a plurality of compressible bladders movably housed in the pocket, each compressible bladder in fluid communication with one of the plurality of pressure sensors.

In the pressure monitor, each of the plurality of pressure sensors may be individually housed in each of the plurality of compressible bladders.

In the pressure monitor, a tube may extend between each of the plurality of pressure sensors and each of the plurality of compressible bladders.

In the pressure monitor, there may be at least six pressure sensors.

The pressure monitor may further comprise at least six vibration motors which are in electronic communication with the microcontroller, the vibration motors proximate to the pressure sensors.

In another embodiment, a method of assessing core strength of a user is provided, the method comprising:
providing a pressure monitor, the pressure monitor including: an adjustable belt, the adjustable belt including an inner surface and an outer surface; a plurality of pressure sensors movably mounted on the inner surface of the adjustable belt; a microcontroller which is mounted on the belt and is in electronic communication with each of the plurality of pressure sensors; and a user interface, the user interface in communication with the microcontroller;
positioning the plurality of pressure sensors over at least a plurality of muscles of the core;
tightening the belt around the user's waist;
the user distending the plurality of muscles of the core;
each pressure sensor sensing a pressure;
the microcontroller receiving a set of data from the plurality of pressure sensors;
and displaying the data set on the user interface.

In the method, the positioning may be effected by sliding the plurality of pressure sensors along the inner surface of the adjustable belt on a slide.

In the method, the positioning may be over four or more of the rectus abdominis, the transverse abdominis, the internal/external obliques, the quadratus lumborum, the erector spinae and the multifidus.

In the method, the positioning may be over the rectus abdominis, the transverse abdominis, the internal and external obliques, the quadratus lumborum, the erector spinae and the multifidus.

In the method, the pressure monitor may further include a plurality of vibration motors in electronic communication with the microcontroller, the microcontroller instructing at least one vibration motor to vibrate over a weak or poorly activated muscle.

### FIGURES

FIG. 1 is a perspective view of the pressure monitoring system of the present technology.

FIG. 2 is a schematic of data collection and display.

FIG. 3 is a perspective view of the pressure monitoring device of the present technology.

FIG. 4A is a plan view of the inner surface of the belt shown in FIG. 1; and FIG. 4B is a cross sectional view of the belt shown in FIG. 1.

FIG. 5 is a block diagram of the method of using the system or device of the present technology.

FIG. 6A is a longitudinal sectional view through the belt of an alternative embodiment belt for the pressure monitoring system and the pressure monitoring device; and FIG. 6B is a cross sectional view through the belt of an alternative embodiment belt for the pressure monitoring system and the pressure monitoring device.

FIG. 7 is a block diagram of the method of using the system or device of the alternative embodiment of FIGS. 6A and 6B.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

The muscles of the abdomen that contribute to core strength and are therefore measured for strength in the present technology include: the rectus abdominis; the transverse abdominis; the internal/external obliques; the quadratus lumborum; the erector spinae; the multifidus; and the diaphragm. The diaphragm is assessed indirectly based on the activity of the other muscles and based on abdominal expansion. The pressure monitor allows one to observe “zones” of outward abdominal pressure consistent with appropriate contractions of the muscles within those zones. Generating appropriate contraction of these muscles will be used to guide and teach the user how to best create ideal lumbopelvic stability/maintain core integrity while exercising.

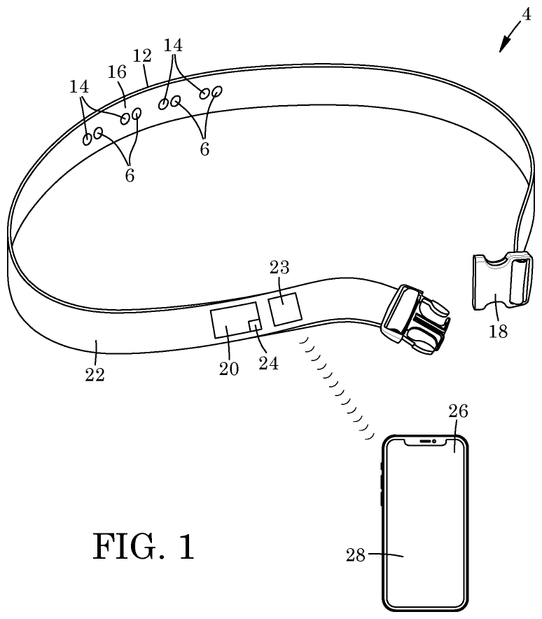

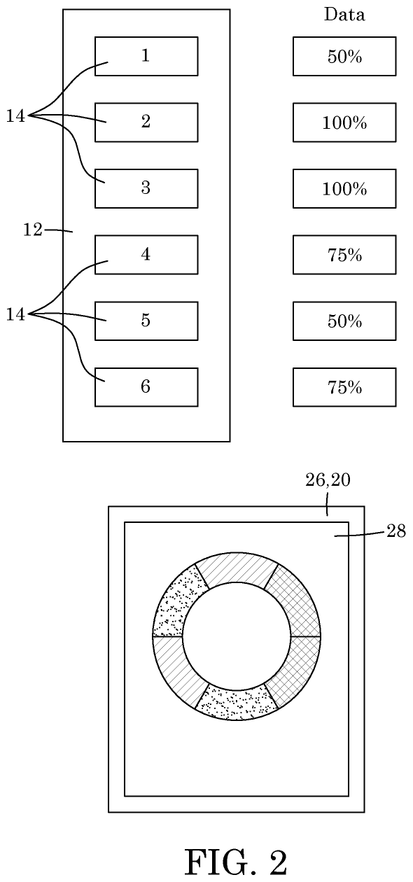

As shown in FIG. 1, a pressure monitoring system, generally referred to as 4, includes a belt 12 with a plurality of mini motor vibration discs 6 and a plurality of pressure sensors 14 spaced apart and located on the inner surface 16 of the belt 12. There are at least six pressure sensors 14, and preferably ten or more. Similarly, there at least six mini motor vibration discs 6 and preferably ten or more. For each pressure sensor 4, there is a mini motor vibration disc 6. The belt 12 is adjustable for different sizes of users and is made from webbing, 400 denier nylon or other material that will conform to the user's waist. The belt 12 may be elastic, inelastic, or a combination thereof. A fastener 18 allows for adjustment of length and may be a buckle or hook and loop (Velcro®). A microcontroller 20 is located on an outer surface 22 of the belt 12 and is positioned to be over top of the rectus abdominis. It is in electronic communication with the pressure sensors 14 and the mini motor vibration discs 6. A battery 23 is in electrical communication with the microcontroller 20 and is releasably retained on the belt 12. The microcontroller 20 includes a wireless radio 24 such as a Bluetooth® radio, for communication with a computing device 26, which may be a Smart phone, a tablet, a desktop computer, a laptop or the like. The computing device 26 includes a user interface 28. The computing device 26 is configured to display pressure sensor data from each pressure sensor 14 on the user interface 28. The display may be numerical, a graph, such as a bar graph or may be a colour coded dial, for example, red for too high pressure, blue for too low pressure and green for optimal pressure, with all the colors in between to provide a continuous range of pressure readouts. There may be individual dials reporting on each pressure sensor or the dial may represent the belt, with each pressure sensor being represented by a segment of the dial. The gold standard intra-abdominal pressure would result in the dial being green around its entirety. As shown in FIG. 2, a user with poor core strength, may, for example, have segments that are blue, segments that are green, segments that are ed and segments of colours therebetween. As described above, the pressure sensors 14 are located around the belt 12 and are aligned above the muscles of interest. The data are processed and are compared to the “gold standard” for a given demographic. The data may be expressed as a percentage of pressure for that muscle in that demographic or any other suitable means of expressing the comparison. The data are displayed on the user interface 28 of the microcontroller 20 or the computing device 26 as a dial or belt with each segment or zone of the dial or belt showing a representation of the processed data. In the case shown in FIG. 2, 50% is shown as hatching (blue), 100% is shown as cross hatching (green) and 75% is shown as stippling (turquoise).

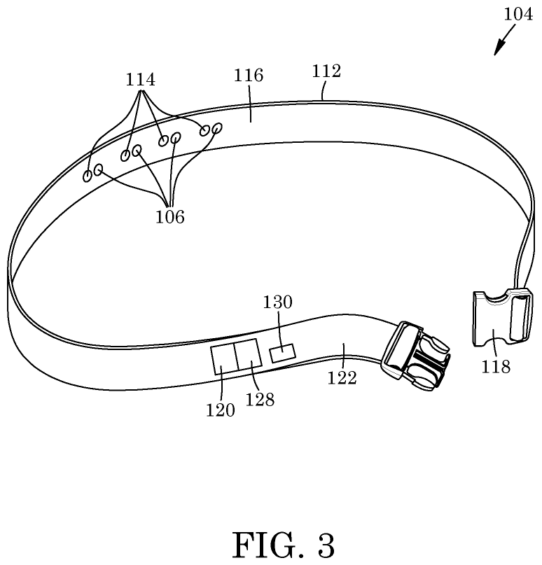

As shown in FIG. 3, in an alternative embodiment, a pressure monitoring apparatus, generally referred to as 104, includes a belt 112 with a plurality of mini motor vibration discs 106 and a plurality of pressure sensors 114 spaced apart and located on the inner surface 16 of the belt 12. There are at least six pressure sensors 114, and preferably ten or more. Similarly, there at least six mini motor vibration discs 106 and preferably ten or more. For each pressure sensor 114, there is a mini motor vibration disc 106. The belt 112 is adjustable for different sizes of users and is made from webbing, 400 denier nylon or other material that will conform to the user's waist. The belt may be elastic, inelastic, or a combination thereof. A fastener 118 allows for adjustment of length and may be a buckle or hook and loop (Velcro®). A microcontroller 120 is located on an outer surface 122 of the belt 112 and is positioned to be over top of the rectus abdominis. It is in electronic communication with the pressure sensors 114 and the mini motor vibration discs 106. The microcontroller 120 is connected to a user interface 128, either electronically, in one embodiment or wirelessly in another embodiment. The microcontroller 120 is configured to display pressure sensor data from each pressure sensor 114 on the user interface 128. A battery 130 is releasably retained on the belt 112 and is in electrical communication with the microcontroller 120. The display may be numerical, a graph, such as a bar graph or may be a colour coded dial, for example, red for high pressure, blue for medium pressure and green for light pressure, with all the colors in between to provide a continuous range of pressure readouts.

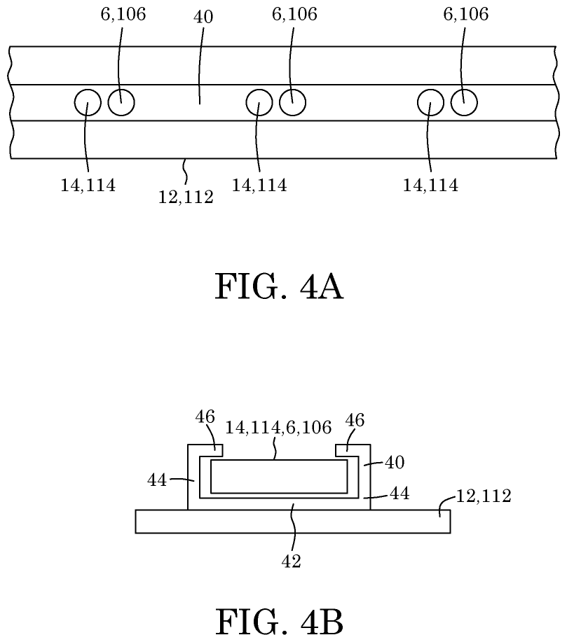

As shown in FIG. 4A, the pressure sensors 14, 114, which are preferably piezo-electric pressure sensors, and the mini motor vibration discs 6, 106 are mounted on a slide 40 which extends a substantial length of the belt 12, 112 on the inner surface 16, 116. The slide 40 allows for correct placement of the pressure sensors 14,114 and the mini motor vibration discs 6, 106 on the user. As shown in FIG. 4B, the slide 40 includes a base 42, sides 44 and a lip 46 on either side to hold the pressure sensor 14, 114 and the mini motor vibration discs 6, 106 in place when in use, but to allow for sliding along the slide 40 when not in use or when positioning the apparatus.

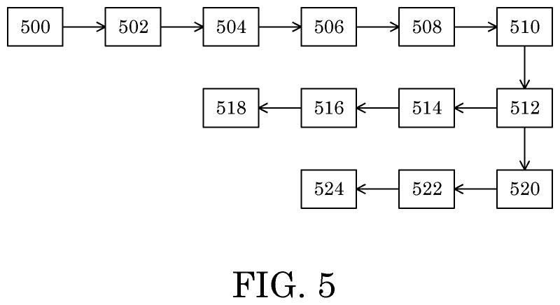

As shown in FIG. 5, the method of using the monitoring device 104 or system 4 to conduct an assessment of comprehensive abdominal strength and breathing mechanics is as follows: the practitioner or user determines 500 the position at least four of the rectus abdominis, the transverse abdominis, the internal/external obliques, the quadratus lumborum, the erector spinae and the multifidus of the patient or user; the pressure sensors and mini motor vibration discs are positioned 502 on the belt such that there will be at least one pressure sensor and its vibration disc for each muscle; the belt is tightened 504 around the patient's or user's waist; the patient or user pushes 506 their core muscles out as much as possible; the pressure sensors sense 508 the pressure of each muscle; the microcontroller receives 510 a signal from each pressure sensor; the microcontroller activates 512 the mini motor vibration disc located over muscles that are weaker or are not activating properly to provide immediate sensory feedback; the microcontroller communicates 514 with the user interface directly; the user interface displays 516 the pressure exerted by each muscle of the core muscles; and the pressure sensor data are stored 518 in the memory; or the microcontroller communicates 520 with a computing device; the computing device displays 522 the pressure exerted by each muscle of the core muscles on the user interface; and the data 524 are stored in the memory. The goal is to teach the user how to create the appropriate amount of intra abdominal pressure throughout their core and thus, give them the best means of stability/lumbopelvic control. By measuring pressure along the length of the belt with the pressure sensors, analyzing the data with the microcontroller or computing device, and providing feedback to the user, the pressure monitor or pressure monitor system provides assessment, feedback and cuing into the individual muscles within the core complex.

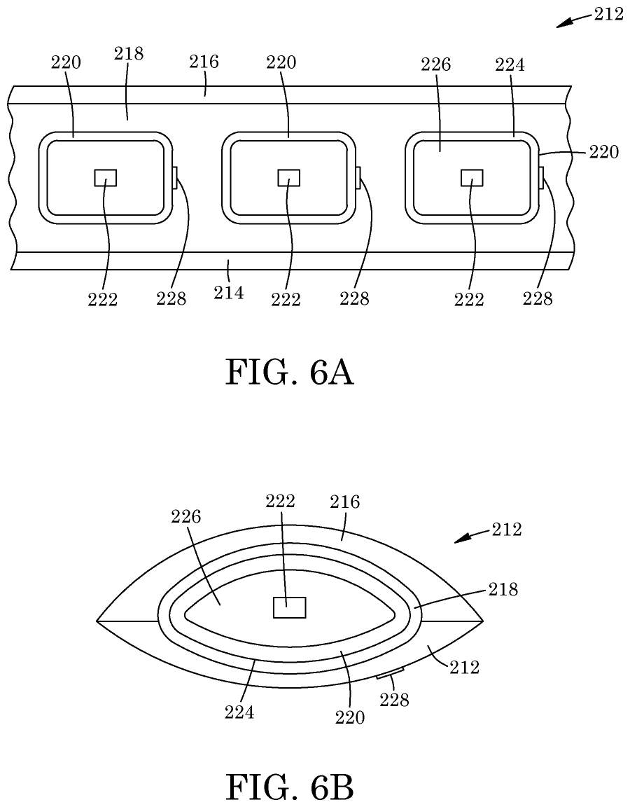

As shown in FIGS. 6A and 6B, in an alternative embodiment, the pressure monitoring system 4 and the pressure monitoring device 104 include a belt, generally referred to as 212, that has an inner layer 214, an outer layer 216 and a pocket 218 therebetween. Within the pocket 218 are a plurality of compressible bladders 220, each with a pressure sensor 222 housed within. There are at least six compressible bladders 220, and preferably ten or more. The compressible bladders 220 have a flexible outer skin 224 and have an interior 226 that contains a fluid such as air, gas, gases or liquid or is a gel. The pressure sensors 222 are either piezo-electric pressure sensors or micro-electromechanical (MEMS) pressure sensors. The compressible bladders 220 are movable within the pocket 218 to ensure proper placement of the compressible bladders 220 and pressure sensors 222. Each compressible bladder 220 has a mini motor vibration disc 228 on the outer skin 224 pressed against the inner layer 214.

In another alternative embodiment, the pressure sensors are in fluid communication with the compressible bladders, one pressure sensor for each compressible bladder and are not housed within the compressible bladders. The pressure sensor 222 is in fluid communication with the interior 226 through a tube.

In another embodiment, there are two rows of sensors 14, 114, an upper row and a lower row and optionally two rows of mini motor vibration discs 6, 106 of one row of mini motor vibration discs 6, 106 which are preferably located between the two rows. The pressure monitoring system 4 and the pressure monitoring device 104 may further include a plurality of four axis accelerometers spaced apart and located on the belt 12, 112 and in electronic communication with the microcontroller 20, 120. There are preferably at least six accelerometers and more preferably ten or more. As an additional or alternative means to adjusting the fit of the belt 12, 112, an inflatable cuff may extend a portion or a length of the belt 12, 112. The inflatable cuff is attached to the inner surface 16, 116 of the belt 12, 112 over the sensors 14, 114 and the mini motor vibration discs 6, 106 or on the outer surface 22, 122 of the belt 12,112. It can be inflated by manually pumping air or mechanically pumping air into the inflatable cuff.

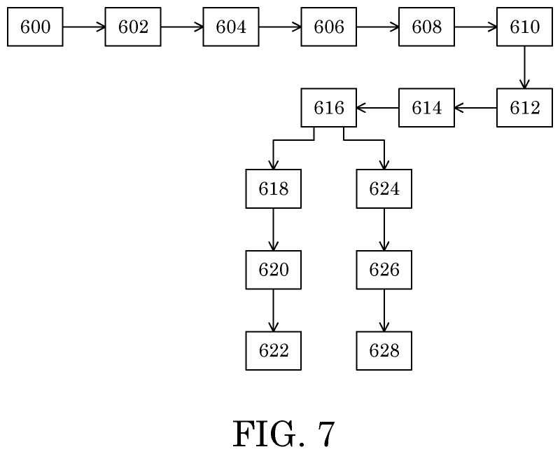

As shown in FIG. 7, the method of using the monitoring device 110 or system 10 with the compressible bladders to conduct an assessment of comprehensive abdominal strength and breathing mechanics is as follows: the practitioner or user determines 600 the position at least four of the rectus abdominis, the transverse abdominis, the internal/external obliques, the quadratus lumborum, the erector spinae and the multifidus of the patient or user; the compressible bladders are positioned 602 on the belt such that there will be at least one compressible bladder with its pressure sensor for each muscle; the belt is tightened 604 around the patient's or user's waist; the patient or user pushes 606 their core muscles out as much as possible (distends the core muscles); the compressible bladders compress 608, exerting pressure 610 on the pressure sensors; the pressure sensors sense 612 the pressure of each muscle through the compression of the compressible bladders; the microcontroller activates 614 the mini motor vibration disc located over muscles that are weaker or are not activating properly to provide immediate sensory feedback the microcontroller receives 616 a signal from each pressure sensor; the microcontroller communicates 618 with the user interface directly to report on the pressures; the user interface displays 620 the pressure exerted by each muscle of the core muscles; and the pressure sensor data are stored 622 in the memory; or the microcontroller communicates 624 with a computing device; the computing device displays 626 the pressure exerted by each muscle of the core muscles on the user interface; and the data are stored 628 in the memory. By measuring pressure along the length of the belt with the pressure sensors, analyzing the data with the microcontroller or computing device, and providing feedback to the user, the pressure monitor or pressure monitor system provides assessment, feedback and cuing into the individual muscles within the core complex.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A pressure monitor for reporting pressure exerted by each of a plurality of core muscles of a user, the pressure monitor comprising: an adjustable belt, the adjustable belt including an inner surface and an outer surface; a plurality of pressure sensors movably mounted on the inner surface of the adjustable belt; a microcontroller which is mounted on the belt and is in electronic communication with each of the plurality of pressure sensors; and a user interface, the user interface in communication with the microcontroller.

2. The pressure monitor of claim 1, wherein the user interface is mounted on the belt.

3. The pressure monitor of claim 2, wherein the microcontroller is configured to receive pressure data from each of the plurality of pressure sensors, display the pressure data on the user interface and store the pressure data.

4. The pressure monitor of claim 1, further comprising a computing device which is in wireless communication with the microcontroller and includes the user interface.

5. The pressure monitor of claim 4, wherein the computing device is configured to receive pressure data from each of the plurality of pressure sensors, display the pressure data on the user interface and store the pressure data.

6. The pressure monitor of claim 5, further comprising a slide on the inner surface of the belt, the plurality of pressure sensors slidably mounted on the slide.

7. The pressure monitor of claim 5, wherein the belt further comprises an inner layer, an outer layer and a pocket therebetween.

8. The pressure monitor of claim 7, further comprising a plurality of compressible bladders movably housed in the pocket, each compressible bladder in fluid communication with one of the plurality of pressure sensors.

9. The pressure monitor of claim 8, wherein each of the plurality of pressure sensors are individually housed in each of the plurality of compressible bladders.

10. The pressure monitor of claim 8, wherein a tube extends between each of the plurality of pressure sensors and each of the plurality of compressible bladders.

11. The pressure monitor of claim 10, wherein there are at least six pressure sensors.

12. The pressure monitor of claim 11, further comprising at least six vibration motors which are in electronic communication with the microcontroller, the vibration motors proximate to the pressure sensors.

13. A method of assessing core strength of a user, the method comprising:
a) providing a pressure monitor, the pressure monitor including: an adjustable belt, the adjustable belt including an inner surface and an outer surface; a plurality of pressure sensors movably mounted on the inner surface of the adjustable belt; a microcontroller which is mounted on the belt and is in electronic communication with each of the plurality of pressure sensors; and a user interface, the user interface in communication with the microcontroller;
b) positioning the plurality of pressure sensors over at least a plurality of muscles of the core;
c) tightening the belt around the user's waist;
d) the user distending the plurality of muscles of the core;
e) each pressure sensor sensing a pressure;
f) the microcontroller receiving a set of data from the plurality of pressure sensors; and
g) displaying the data set on the user interface.

14. The method of claim 13, wherein the positioning is effected by sliding the plurality of pressure sensors along the inner surface of the adjustable belt on a slide.

15. The method of claim 14, wherein the positioning is over four or more of the rectus abdominis, the transverse abdominis, the internal/external obliques, the quadratus lumborum, the erector spinae and the multifidus.

16. The method of claim 15, wherein the positioning is over the rectus abdominis, the transverse abdominis, the internal and external obliques, the quadratus lumborum, the erector spinae and the multifidus.

17. The method of claim 16, wherein the pressure monitor further includes a plurality of vibration motors in electronic communication with the microcontroller, the microcontroller instructing at least one vibration motor to vibrate over a weak or poorly activated muscle.
